(19) United States
(12) Plant Patent Application Publication
(10) Pub. No.: US 2005/0246807 P1
(43) Pub. Date: Nov. 3, 2005

(54) Carnation plant called 'CFPC Yukata'

(50) DIANTHUS CARYOPHYLLUS; CARNATION

(76) Inventor: Oscar T. Hasegawa, Salinas, CA (US)

Correspondence Address: CHRISTIE, PARKER & HALE, LLP, PO BOX 7068, PASADENA, CA, 91109-7068, US

(21) Appl. No.: 10/836,951

(22) Filed: Apr. 30, 2004

(51) Int. Cl. A01H 5/00

(52) U.S. Cl. PLT/273

(57) ABSTRACT

A new and distinct variety of carnation plant characterized by the white and yellow background base color with purple streaks and elongated spots radiating from the center of the flower.

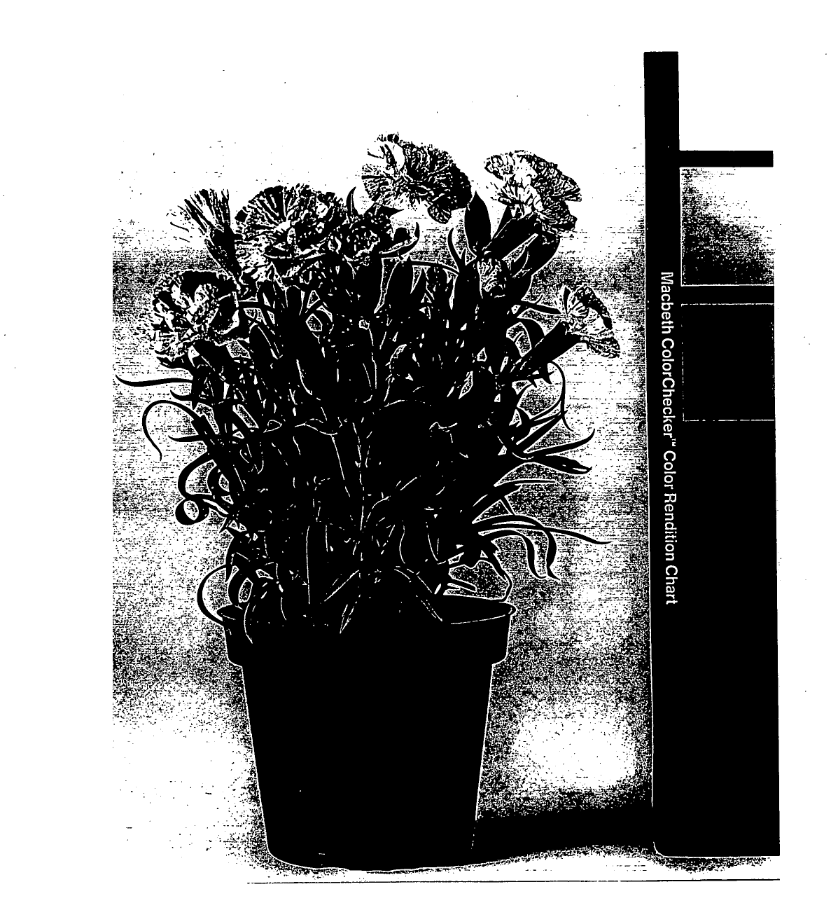

### BOTANICAL CLASSIFICATION

The present invention relates to a new and distinct variety of hybrid Dianthus caryophyllus. The varietal denominator of the new variety is ‘CFPC Yukata’.

### BACKGROUND OF THE INVENTION

The present invention relates to a new and distinct variety of carnation plant, named ‘CFPC Yukata’, which was originated by me from a cross made in a controlled breeding program. The female, or seed, parent is unpatented and identified as 990275; the male, or pollen, parent is unknown. The color of the female parent's flower is dark pink with hot pink picotee. The primary objective of the controlled breeding program was to produce a cultivar having a flower of new color.

### SUMMARY OF THE INVENTION

Among the novel characteristics possessed by the new variety which distinguishes it is a distinctive yellow and white background with radial solid and broken streaks of purple flower color. The present inventor is unaware of any other pot carnation commercially available in this color combination. When grown in 4½ inch plastic pots in Salinas, Calif., plant diameter averages 6½ inch. Pinched plants are about 7 inches tall and flowers are about 1⅞ inches average diameter with a ⅝ inch crown (average) Foliage is about 3¼ inches long and about ⅜ inches wide.

Asexual reproduction by propagation by side shoot cuttings of the new variety as performed in Salinas, Calif., shows that the foregoing and other distinguishing characteristics come true to form and are established and transmitted through succeeding propagations.

### BRIEF DESCRIPTION OF ILLUSTRATION

The accompanying photographic illustration shows a typical specimen of the new variety in color as nearly true as it is reasonably possible to make in a color illustration of this character.

### DESCRIPTION OF THE NEW VARIETY

Referring now specifically to the new and distinct variety of carnation plant the following is a detailed description of ‘CFPC Yukata’ with color description where indicated by reference to The Royal Horticultural Society Colour Chart, except where common terms of color definition are employed. Color designation and other values stated may deviate slightly from the stated values from flowering to flowering, but the deviations will be within the range expected from varying environmental, seasonal and cultural conditions.

The new variety of carnation plant as herein described may vary in slight detail due to climatic, soil and cultural conditions under which the variety may be grown, the present description being of the variety as grown inside flower testing facilities at Salinas, Calif. The height of the plant and flower and foliage size were all established as an average for a 118-day old plant using standard commercial cultural practices. The data accurately characterizes the new cultivar in question.

The new variety is a greenhouse-type plant, suitable for production as a pot spray.
Plant:
   Height.—About 7 inches tall.
   Growing habit.—Semi-upright.
   Blooming habit.—As a spray when grown in a pot.
   Rooting time.—About 18 days.
   Breaking action.—Good.
   Mutation prone.—No.
   Disease Resistance.—Good.
Foliage:
   Color.—Upper side: near 136A; lower side: near 137A.
   Size.—Length: about 3 14 inches; width: about ⅜ inches.
   Stem color.—Near 139B.
   Stem diameter.—Base: about 3/16 inches;
   Apex: about 1/16 inches.
   Leaf base.—Color: near 148D; Shape: smooth edge.
   Leaf apex.—Color: near 136A; Shape: smooth edge, lanceolate.
Bud:
   Shape.—Ovoid.
   Color.—Near 144B.
   Size.—length: about 1⅛ inches; diameter: about ⅜ inches.
Calyx:
   No. of lobes (sepals).—Five.
   Size.—Length: about 5/16 inches; width: about 3/16 inches.
   Color.—Outside: near 144B; inside: near 148D.
Bloom:
   Size.—Diameter (average): about 1⅞ inches; depth (average): about ⅝ inches.
   Borne.—As a spray.
   Color.—General tonality: yellow and white background with radial solid and broken streaks of purple; Upper side: near 155D (whiter than 155D), 8C and 78A; Lower side: near 155D (whiter than 155D), 8C and 78A; Center: near 145C; Outer petals: near 155D (whiter than 155D), 8C and 78A.
   Keeping quality.—Good.
   Fragrance.—Strong clove.
Petals:
   Number.—Twenty six.
   Outer petals.—Length: about 1⅞ inches; Width: about 1 1/16 inches.
   Central petals.—Length: about 1½ inches; Width: about ¾ inches.
   Texture.—Smooth.
   Appearance.—Shiny.
   Form.—Fan-shaped.
   Edge.—Smooth to very slight serration.
Reproductive organs:
   Stamen.—None (male sterile).
   Anthers.—None.
   Filaments.—None.
   Pistil.—Length: 1 1/16 inches; Number: one (bilobed) Color: White (whiter than 155D).
   Stigma.—Number: 2; Color: White (whiter than 155D).
   Fertility.—Male: sterile; Female: Good.
   Ovary.—Shape: conical; Color: base near 145D, body near 145B, apex near 145C.
   Size.—Length: about 5/16 inches; Width: about 3/16 inches.
Fruit and seed: None produced.

1. A new and distinct variety of carnation plant identified as ‘CFPC Yukata’, substantially as shown and described.
